(19) United States
(12) Patent Application Publication
(10) Pub. No.: US 2011/0112451 A1
(43) Pub. Date: May 12, 2011

(54) SYSTEM AND METHOD FOR LIMITING BODY MOTION USING EXOSKELETAL PIECES COUPLED WITH LIMITING CONNECTORS

(76) Inventor: Edward D. Miller, Legrand, CA (US)

(21) Appl. No.: 12/943,867

(22) Filed: Nov. 10, 2010

Related U.S. Application Data
(60) Provisional application No. 61/260,319, filed on Nov. 11, 2009.

(51) Int. Cl. A61F 5/00

(52) U.S. Cl. 602/5; 128/846; 602/27

(57) ABSTRACT

Disclosed are a system and method for limiting body or joint motion using exoskeletal pieces coupled with limiting connectors. In an exemplary system, a plurality of exoskeletal pieces are attached to various positions on a wearer's body proximate to a joint or body location on or within the wearer's body. The joint or body location is selected as requiring limited motion based on, for example, the goal of avoiding causing or exacerbating bodily injury. Once so attached, limiting connectors coupling the plurality of exoskeletal pieces allow free motion or natural motion in one or more degrees of freedom of the joint or body location within certain limits, but prevent free motion outside those limits.

### CROSS-REFERENCE TO RELATED APPLICATIONS

This application claims the benefit of U.S. Provisional Application No. 61/260,319 entitled “SYSTEM AND METHOD FOR LIMITING BODY MOTION USING EXOSKELETAL PIECES COUPLED WITH LIMITING CONNECTORS”, filed Nov. 11, 2009, and is hereby incorporated by reference in its entirety.

### BACKGROUND INFORMATION

Methods and systems for the prevention and correction of injury have received considerable study and refinement, because such methods and systems are important in a wide range of human endeavors. Every endeavor which entails some risk to health has received at least some such study. One exemplary endeavor, sports, will be discussed below.

Modern sports are commercially and personally significant activities into which many people invest significant amounts of money and time. Thus, much effort has gone into improving methods and systems for the prevention and correction of sports injuries, because sports injuries tend to reduce the profitability and personal satisfaction that result from sporting. Sports injuries include, for example, traumatic injuries and overuse injuries. Sports injuries further include contusions, muscle strains, tendon injuries, ligament sprains, etc. A sprained ankle, which is a ligament sprain occurring around certain joints and in certain ligaments in and above the foot, is one exemplary sports injury.

Despite being a particularly pernicious sports injury, the methods and systems for preventing and correcting sprained ankles, and other joint-related ligament injuries, are still somewhat wanting. One method for preventing a sprained ankle includes wrapping an athlete's foot, ankle, and lower leg in medical tape prior to a sports activity. Such a method is inexpensive, but tends to unduly restrain the taped athlete's mobility, and can cause discomfort. The same taping method, when applied after an ankle is sprained to help correct the sprained ankle, is similarly unduly restraining and uncomfortable.

A system for preventing joint injuries that improves somewhat on the taping method involves attaching an exterior artificial hinge over the location of the injury with a pair of braces on opposite sides of the location. For example, to help prevent or correct a knee injury, braces are attached to the upper leg and to the lower leg, and an artificial hinge is coupled between the braces proximate to the knee. Working together, the braces and artificial hinge constrain motion of the knee joint to motion around one axis (i.e. the axis of the artificial hinge). This constraint helps prevent or correct knee injury, but like the taping method, can also lead to significantly constrained mobility.

Such methods and systems in the prior art suffer from a variety of drawbacks including and beyond those described above. Thus, improved methods and systems are required.

### BRIEF DESCRIPTION OF THE DRAWINGS

FIG. 1A depicts a side view of an example system for limiting body or joint motion with respect to an ankle joint.

FIG. 1B depicts a rear view of an example system for limiting body or joint motion with respect to an ankle joint.

FIG. 1C depicts a rear view of an example system for limiting body or joint motion with respect to an ankle joint about which a foot is rotated or moved.

FIG. 2A depicts a side view of an example system for limiting body or joint motion with respect to an elbow joint.

FIG. 2B depicts a rear view of an example system for limiting body or joint motion with respect to an elbow joint.

FIG. 3 depicts a flowchart of a method for limiting body or joint motion according to one embodiment of the present invention.

### DESCRIPTION OF THE INVENTION

Disclosed are a system and method for limiting body or joint motion using exoskeletal pieces coupled with limiting connectors. In an exemplary system, a plurality of exoskeletal pieces are attached to various positions on a wearer's body proximate to a joint or body location on or within the wearer's body. The joint or body location is selected as requiring limited motion based on, for example, the goal of avoiding causing or exacerbating bodily injury. Once so attached, limiting connectors coupling the plurality of exoskeletal pieces allow free motion or natural motion in one or more degrees of freedom of the joint or body location within certain limits, but prevent free motion outside those limits.

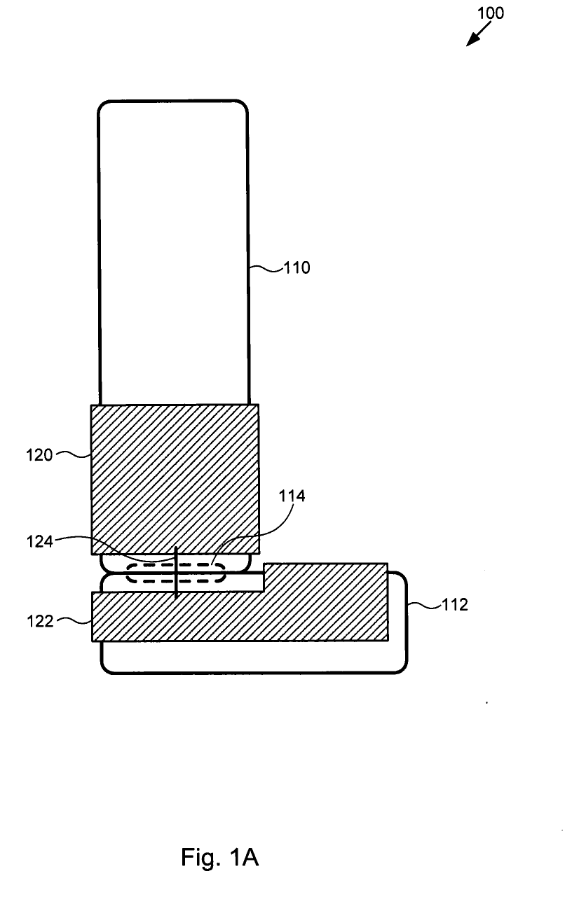

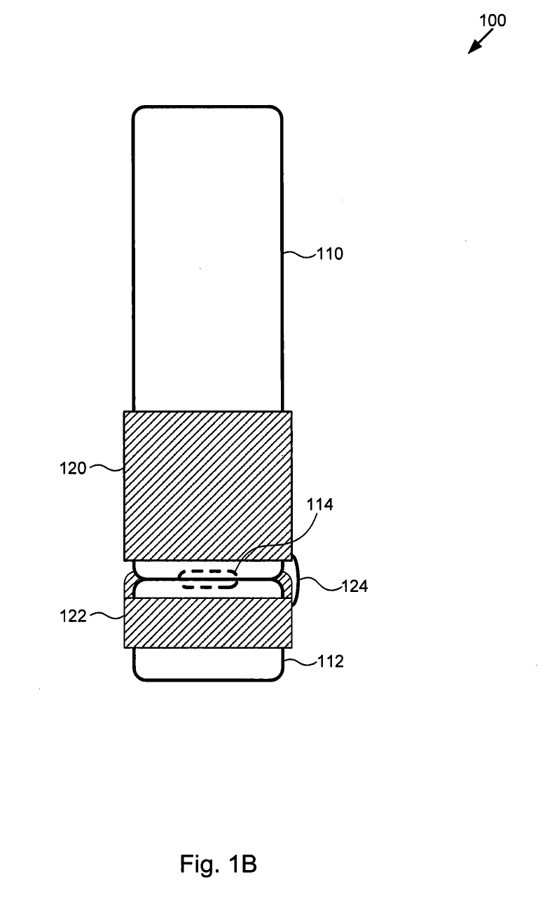

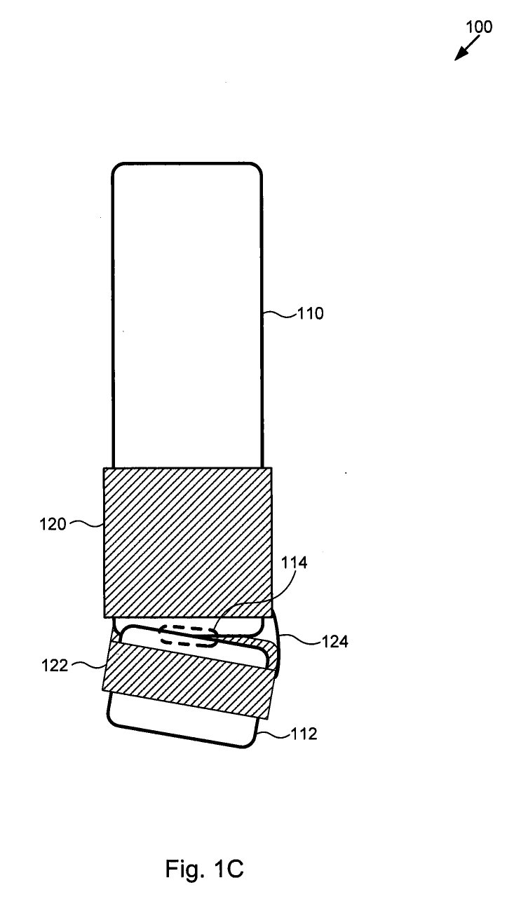

FIG. 1A depicts system 100 for limiting body or joint motion using exoskeletal pieces coupled with a limiting connector according to one embodiment of the invention. System 100 includes exoskeletal piece 120, exoskeletal piece 122, and limiting connector 124. Exoskeletal piece 120 is depicted as attached to leg 110, and exoskeletal piece 122 is depicted as attached to foot 112. Leg 110 and foot 112 are part of the body of a wearer of system 100. The remainder of the body of the wearer is not depicted in FIG. 1A, but it should be understood that the wearer can be an athlete, a medical patient, etc. Between leg 110 and foot 112 is ankle joint 114, which allows for natural motion of foot 112 with respect to leg 110 as is known in the art. Ankle joint 114 is an example of a protected body location of system 100.

As depicted in FIG. 1A, exoskeletal piece 120 is attached to leg 110. Although exoskeletal piece 120 is depicted as a solid piece in FIG. 1A, in various embodiments exoskeletal piece 120 can include several sub-pieces. For example, in one embodiment exoskeletal piece 120 includes a shin piece and a back piece that are laced or velcroed together to be attached to leg 110 by the wearer. In another embodiment, exoskeletal piece 120 is a part of a pants-leg or a part of a boot, for example. In yet another embodiment, exoskeletal piece 120 is a shoe piece attached around leg 110.

As further depicted in FIG. 1A, exoskeletal piece 122 is attached to foot 112. Although exoskeletal piece 122 is depicted as a solid piece in FIG. 1A, in various embodiments exoskeletal piece 122 can include several sub-pieces. For example, in one embodiment exoskeletal piece 122 includes a sole piece and a top piece that are laced or velcroed together to be attached to foot 112 by the wearer. In another embodiment, exoskeletal piece 122 is a shoe piece attached around foot 112.

FIG. 1B depicts another view of system 100. While FIG. 1A depicts a side-view of system 100, FIG. 1B depicts a rear view. Limiting connector 124 is depicted in both FIG. 1A and FIG. 1B as coupled to exoskeletal piece 120 and exoskeletal piece 122. In particular, in one embodiment a first endpoint of limiting connector 124 is coupled to exoskeletal piece 120, and a second endpoint of limiting connector 124 is coupled to exoskeletal piece 122. In various embodiments of the present invention, the endpoints of limiting connector 124 can be coupled in various ways.

The endpoints can be, for example, tied to, laced to, velcroed to, glued to, or magnetically coupled to exoskeletal piece 120 and exoskeletal piece 122. Further, the endpoints can be, for example, fixedly coupled to or adjustably coupled to exoskeletal piece 120 and exoskeletal piece 122. In these various embodiments, the wearer of system 100 has the ability to adjust system 100 to suit his particular physiology or otherwise suit his preference. Further still, exoskeletal piece 120 and exoskeletal piece 122 themselves can be adjustable to accommodate the wearer. For example, exoskeletal piece 120 can be tightened, loosened, raised, or lowered with respect to leg 110. Similarly, exoskeletal piece 122 can be tightened, loosened, lengthened, or shortened with respect to foot 112.

Besides being adjustable by the wearer of system 100, the variables stated above can also be adjusted by a doctor or other health technician. For example, during an athletic event an athlete may require immediate use of an embodiment of the present invention, but may be untrained in its use. Consequently, a sports medicine professional, properly trained in the use and adjustment of system 100, can apply system 100 to the athlete in order to prevent or to help manage an injury. In another embodiment, an athlete or other intended wearer of system 100 can visit a doctor's office or other health facility seeking medical advice or aide, and receive a professional fitting of system 100 by the doctor or the health professional. Such professional fitting may include, for example, a detailed examination of the wearer's injury or physiology to fine-tune particular variables of system 100 to suit the wearer's injury or physiology.

One characteristic of limiting connector 124 is that the second endpoint of limiting connector 124 is configured to move freely with respect to the first endpoint of limiting connector 124 within a limiting factor of limiting connector 124. Because of this characteristic, natural motion of ankle joint 114, the protected body location, is allowed by system 100 within the limiting factor. However, because of the limiting factor of limiting connector 124, ankle joint 114 cannot move freely outside the limiting factor of limiting connector 124.

The effect of the limiting factor of limiting connector 124 in one embodiment is illustrated by a comparison of FIG. 1B with FIG. 1C. Both FIG. 1B and FIG. 1C depict a rear view of system 100. Whereas in FIG. 1B, foot 112 is not depicted as rotated or moved about ankle joint 114, in FIG. 1C foot 112 is so rotated. Specifically, FIG. 1C depicts foot 112 and exoskeletal piece 122 rotated about ankle joint 114. In another sense, FIG. 1C depicts foot 112 and exoskeletal piece 122 rotated with respect to leg 110 and exoskeletal piece 120. Whereas there was slack in limiting connector 124 as depicted in FIG. 1B, there is no slack in limiting connector 124 as depicted in FIG. 1C. This is the case because, for example, the rotation around ankle joint 114 has caused limiting connector 124 to extend to the limit of its length. Any further rotation around ankle joint 114 is prevented, because limiting connector 124 cannot be lengthened further. Thus, the rotation or movement depicted in FIG. 1C is a natural movement of ankle joint 114, and is prevented from moving outside the limiting factor of limiting connector 124.

In one embodiment, limiting connector 124 as depicted in FIG. 1A, FIG. 1B, and FIG. 1C is implemented as a link, such as a rope, a cable, or a cord. Further, limiting connector 124 can be a natural rope, a plastic link, a metal line, a synthetic member, etc. Limiting connector 124 is, in one embodiment, substantially inelastic. In such an embodiment, an extension of limiting connector 124 as depicted in FIG. 1C is a maximum extension and ankle joint 114 cannot rotate further. In another embodiment, limiting connector 124 is substantially elastic. In such an alternative embodiment, an extension of limiting connector 124 as depicted in FIG. 1C is not a maximum extension, and ankle joint 114 can rotate further, albeit in a restricted fashion as the elastic resistance of limiting connector 124 must be overcome.

As depicted in FIG. 1A, FIG. 1B, and FIG. 1C, the limiting factor of limiting connector 124 is a length of limiting connector 124. However, in another embodiment the limiting factor can be a different characteristic of limiting connector 124. Thus, in one embodiment the limiting factor is a plane in which limiting connector 124 lays. In another embodiment, the limiting factor is a volume. In various embodiments the limiting factor can be influenced by, for example, the thickness, the proximity to the protected body area, the temperature, or another mechanical characteristic of limiting connector 124.

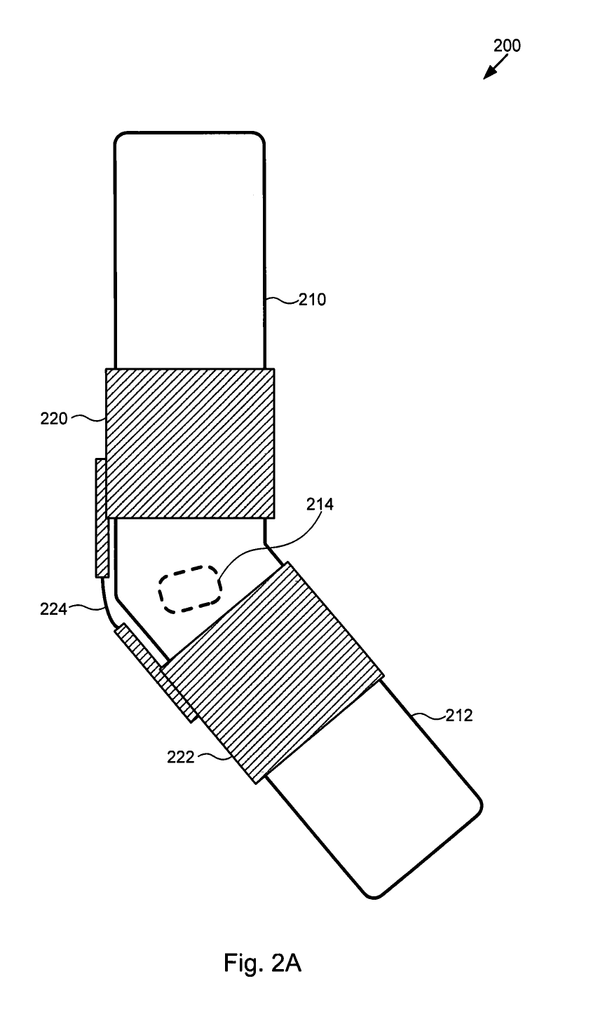

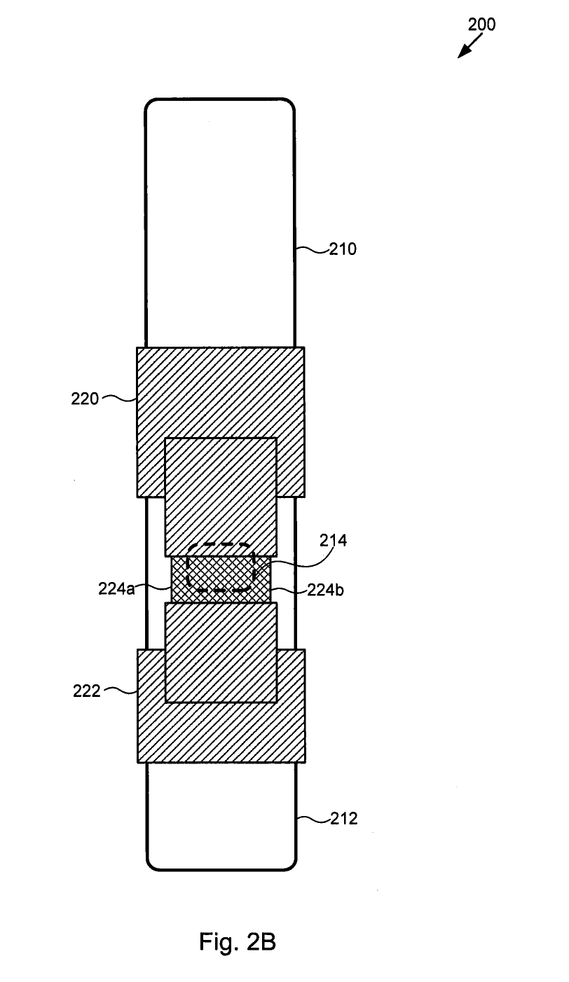

FIG. 2A depicts system 200 for limiting body or joint motion using exoskeletal pieces coupled with a limiting connector according to one embodiment of the invention. System 200 includes exoskeletal piece 220, exoskeletal piece 222, and limiting connector 224. Exoskeletal piece 220 is depicted as attached to upper arm 210, and exoskeletal piece 222 is depicted as attached to lower arm 212. Upper arm 210 and lower arm 212 are part of the body of a wearer of system 200. The remainder of the body of the wearer is not depicted in FIG. 2A, but it should be understood that the wearer can be an athlete, a medical patient, etc. Between upper arm 210 and lower arm 212 is elbow joint 214, which allows for natural motion of lower arm 212 with respect to upper arm 210 as is known in the art. Elbow joint 214 is an example of a protected body location of system 100.

As depicted in FIG. 2A, exoskeletal piece 220 is attached to upper arm 210. In particular, exoskeletal piece 220 is depicted as including a sleeve portion that fits around upper arm 210, and a mounting portion for attachment to limiting connector 224. The mounting portion serves to locate limiting connector 224 proximate to a protected body location. Although exoskeletal piece 220 is depicted as a solid piece in FIG. 2A, in various embodiments exoskeletal piece 220 can include several sub-pieces. For example, in one embodiment exoskeletal piece 220 includes a front piece and a back piece that are laced or velcroed together to be attached to upper arm 210 by the wearer. In another embodiment, exoskeletal piece 220 is a part of a shirt sleeve, for example.

As further depicted in FIG. 2A, exoskeletal piece 222 is attached to lower arm 212. In particular, exoskeletal piece 222 is depicted as including a sleeve portion that fits around lower arm 212, and a mounting portion for attachment to limiting connector 224. The mounting portion serves to locate limiting connector 224 proximate to a protected body location. Although exoskeletal piece 222 is depicted as a solid piece in FIG. 2A, in various embodiments exoskeletal piece 222 can include several sub-pieces. For example, in one embodiment exoskeletal piece 222 includes a front piece and a back piece that are laced or velcroed together to be attached to lower arm 212 by the wearer. In another embodiment, exoskeletal piece 222 is a part of a shirt sleeve.

FIG. 2B depicts another view of system 200. While FIG. 2A depicts a side-view of system 200, FIG. 2B depicts a rear view. Limiting connector 224 of FIG. 2A is depicted in FIG. 2B as having limiting connector edge 224a and limiting connector edge 224b coupled to exoskeletal piece 220 and exoskeletal piece 222. Notably, limiting connector edge 224a and limiting connector edge 224b are, for example, two edges of a sheet. Thus, in one embodiment a first endpoint of limiting connector edge 224a is coupled to exoskeletal piece 220, and a second endpoint of limiting connector edge 224a is coupled to exoskeletal piece 222, while a first endpoint of limiting connector edge 224b is also coupled to exoskeletal piece 220, and a second endpoint of limiting connector edge 224b is also coupled to exoskeletal piece 222. Notably, in one embodiment, the sheet is connected to exoskeletal piece 220 and exoskeletal piece 222 with continuous connections, i.e. with a continuous connection between the first endpoints of limiting connector edge 224a and limiting connector edge 224b, and with a continuous connection between the second endpoints of limiting connector edge 224a and limiting connector edge 224b.

The endpoints can be, for example, tied to, laced to, velcroed to, glued to, or magnetically coupled to exoskeletal piece 220 and exoskeletal piece 222. Further, the endpoints can be, for example, fixedly coupled to or adjustably coupled to exoskeletal piece 220 and exoskeletal piece 222. In these various embodiments, the wearer of system 200 has the ability to adjust system 200 to suit his particular physiology or otherwise suit his preference. Further still, exoskeletal piece 220 and exoskeletal piece 222 themselves can be adjustable to accommodate the wearer. For example, exoskeletal piece 220 can be tightened, loosened, raised, or lowered with respect to upper arm 210. Similarly, exoskeletal piece 222 can be tightened, loosened, raised, or lowered with respect to lower arm 212. Further, a sleeve part and a mounting part of either exoskeletal piece 220 or exoskeletal piece 222 can be adjusted with respect to each other.

Besides being adjustable by the wearer of system 200, the variables stated above can also be adjusted by a doctor or other health technician. For example, during an athletic event an athlete may require immediate use of an embodiment of the present invention, but may be untrained in its use. Consequently, a sports medicine professional, properly trained in the use and adjustment of system 200, can apply system 200 to the athlete in order to prevent or to help manage an injury. In another embodiment, an athlete or other intended wearer of system 200 can visit a doctor's office or other health facility seeking medical advice or aide, and receive a professional fitting of system 200 by the doctor or the health professional. Such professional fitting may include, for example, a detailed examination of the wearer's injury or physiology to fine-tune particular variables of system 200 to suit the wearer's injury or physiology.

One characteristic of limiting connector 224 is that the various endpoints of limiting connector 224 are configured to move freely with respect to each other within a limiting factor of limiting connector 224. Because of this characteristic, natural motion of elbow joint 214, the protected body location, is allowed by system 200 within the limiting factor. However, because of the limiting factor of limiting connector 224, elbow joint 214 cannot move freely outside the limiting factor of limiting connector 224.

The effect of the limiting factor of limiting connector 224 in one embodiment is illustrated by an example. In FIG. 2A, depicting a side view of system 200, it is clear that as the wearer bends his elbow (i.e. bends elbow joint 214), the slack will be removed from the sheet of limiting connector 214 until limiting connector 214 is taut. At such point, no further elbow motion is possible, but prior to such point, the elbow can move freely and naturally. In FIG. 2B, depicting a rear view of system 200, additional detail can be explained.

In particular, in FIG. 2B, if elbow joint 214 is bent and twisted, or rotated, by the wearer, one side of limiting connector 224 will slacken and the other side will become tight. For example, with a clockwise rotation of lower arm 212 with respect to upper arm 210, limiting connector edge 224a can become taut while limiting connector edge 224b can slacken, and vice versa. This alternate slacking and tightening serves to allow natural motion of elbow joint 214 within a limiting factor of limiting connector 224, where the particular limiting factor in this case is determined by both limiting connector edge 224a and limiting connector edge 224b. When the natural motion exceeds the limiting factor, the motion is arrested to protect elbow joint 214.

In one embodiment, limiting connector 224 as depicted in FIG. 2A and FIG. 2B is implemented as multiple links, such as ropes, cables, or cords. Further, limiting connector 224 can be a natural, fabric, plastic, or metallic mesh, or sheet. Limiting connector 224 is, in one embodiment, substantially inelastic. In such an embodiment, an extension of limiting connector edge 224a or limiting connector edge 224b is a maximum extension, and elbow joint 214 cannot rotate or bend further. In another embodiment, limiting connector 224 is substantially elastic. In such an alternative embodiment, elbow joint 214 can rotate further, albeit in a restricted fashion as the elastic resistance of limiting connector 224 must be overcome.

As depicted in FIG. 2A and FIG. 2B, the limiting factor of limiting connector 224 is a set of lengths of limiting connector 224. However, in another embodiment the limiting factor can be a different characteristic of limiting connector 224. Thus, in one embodiment the limiting factor is a plane in which limiting connector 224 lays. In another embodiment, the limiting factor is a volume. In various embodiments the limiting factor can be influenced by, for example, the thickness, the proximity to the protected body area, the temperature, or another mechanical characteristic of limiting connector 224.

Notably, although system 100 in FIG. 1A is explained above as depicted as proximate to ankle joint 114, and although system 200 in FIG. 2A is explained above as depicted as proximate to elbow joint 214, other embodiments of the present invention are not limited to ankle joint or elbow joint protected body locations. For example, system 100 or system 200 could in some embodiments protect a laceration, a burn, a surgery, a scar tissue, or a skin condition. Further, system 100 or system 200 could protect foot skin, leg skin, torso skin, abdominal skin, arm skin, hand skin, or head skin. Further still, system 100 or system 200 could protect shallow tissue, dermal tissue, or fat tissue. Further, system 100 or system 200 could protect a muscle injury, a tendon injury, a bone injury, a ligament injury, a nerve injury, a surgery, or an internal tissue condition. Yet further, system 100 or system 200 could protect a toe joint, an ankle joint, a knee joint, a pelvic joint, a spinal joint, a torso joint, a shoulder joint, an elbow joint, a wrist joint, a finger joint, a neck joint, or a jaw joint. Thus, the method and system disclosed herein is suitable for protecting many different protected body locations, of either human bodies or the bodies of other species, and the examples herein are merely illustrative.

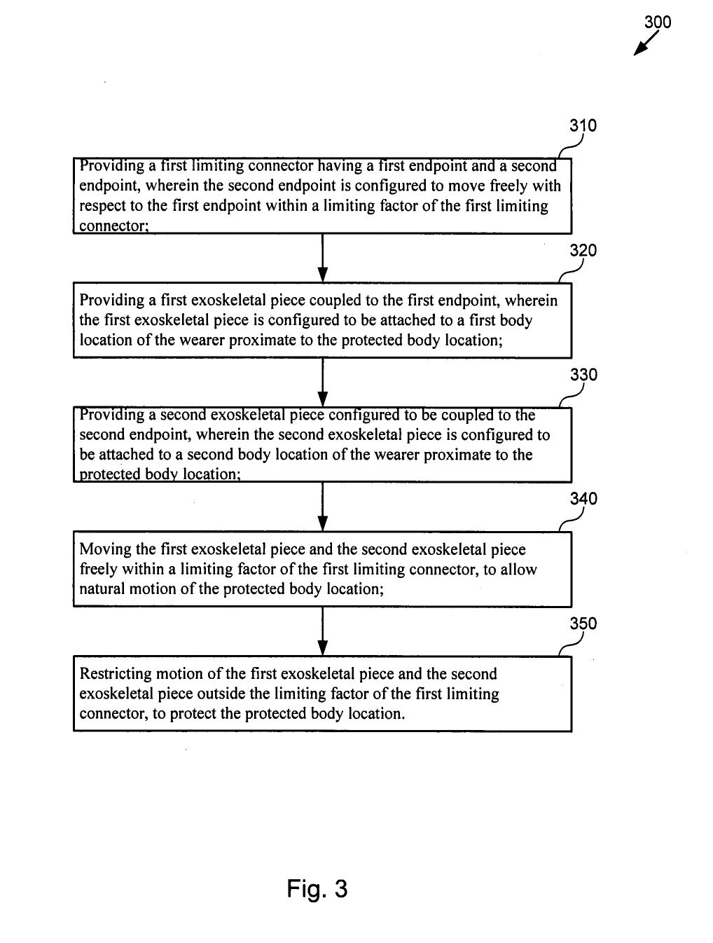

FIG. 3 depicts flowchart 300 for limiting body or joint motion using exoskeletal pieces coupled with limiting connectors according to one embodiment of the invention. Step 310 includes providing a first limiting connector having a first endpoint and a second endpoint, wherein the second endpoint is configured to move freely with respect to the first endpoint within a limiting factor of the first limiting connector. Step 320 includes providing a first exoskeletal piece coupled to the first endpoint, wherein the first exoskeletal piece is configured to be attached to a first body location of the wearer proximate to the protected body location. Step 330 includes providing a second exoskeletal piece configured to be coupled to the second endpoint, wherein the second exoskeletal piece is configured to be attached to a second body location of the wearer proximate to the protected body location. Step 340 includes moving the first exoskeletal piece and the second exoskeletal piece freely within a limiting factor of the first limiting connector, to allow natural motion of the protected body location. Finally, step 350 includes restricting motion of the first exoskeletal piece and the second exoskeletal piece outside the limiting factor of the first limiting connector, to protect the protected body location.

What is claimed is:

1. A system for limiting body motion of a protected body location of a wearer, the system comprising:
a first limiting connector having a first endpoint and a second endpoint, wherein the second endpoint is configured to move with respect to the first endpoint within a limiting factor of the first limiting connector;
a first exoskeletal piece coupled to the first endpoint, wherein the first exoskeletal piece is configured to be attached to a first body location of the wearer proximate to the protected body location; and
a second exoskeletal piece configured to be coupled to the second endpoint, wherein the second exoskeletal piece is configured to be attached to a second body location of the wearer proximate to the protected body location;
wherein the first exoskeletal piece and the second exoskeletal piece move within a limiting factor of the first limiting connector, to allow natural motion of the protected body location, and wherein the first exoskeletal piece and the second exoskeletal piece cannot move outside the limiting factor of the first limiting connector, to protect the protected body location.

2. The system of claim 1, wherein the first limiting connector includes a link.

3. The system of claim 1, wherein the first limiting connector includes a sheet.

4. The system of claim 1, wherein the first limiting connector is substantially elastic.

5. The system of claim 1, wherein the first limiting connector is substantially inelastic.

6. The system of claim 1, wherein the limiting factor is a length of the first limiting connector.

7. The system of claim 1, wherein the limiting factor is a plane in which the first limiting connector lays.

8. The system of claim 1, wherein the limiting factor is a volume.

9. The system of claim 1, wherein moving within a limiting factor of the first limiting connector includes moving within a plurality of degrees of freedom.

10. The system of claim 1, wherein the protected body location is a surface region of the body of the wearer.

11. The system of claim 10, wherein the first body location and the second body location are on substantially opposite sides of the protected body location.

12. The system of claim 10, wherein the surface region is protected from a condition selected from the group consisting of a laceration, a burn, a surgery, a scar tissue, and a skin condition.

13. The system of claim 10, wherein the protected body location is selected from the group consisting of foot skin, leg skin, torso skin, abdominal skin, arm skin, hand skin, and head skin.

14. The system of claim 10, wherein the protected body location is selected from the group consisting shallow tissue, dermal tissue, and fat tissue.

15. The system of claim 1, wherein the protected body location is an interior region of the body of the wearer.

16. The system of claim 15, wherein the first body location and the second body location are on substantially opposite sides of an epicenter of the protected body location.

17. The system of claim 15, wherein the interior region is protected from a condition selected from the group consisting of a muscle injury, a tendon injury, a bone injury, a ligament injury, a nerve injury, a surgery, and an internal tissue condition.

18. The system of claim 15, wherein the protected body location is selected from the group consisting of a toe joint, an ankle joint, a knee joint, and a pelvic joint.

19. A system for limiting body motion of an ankle joint of a wearer, the system comprising:
a first limiting connector having a first endpoint and a second endpoint, wherein the second endpoint is configured to move with respect to the first endpoint within a limiting factor of the first limiting connector;
a first exoskeletal piece coupled to the first endpoint, wherein the first exoskeletal piece is configured to be attached to a first body location on the leg of the wearer proximate to the ankle joint; and
a second exoskeletal piece configured to be coupled to the second endpoint, wherein the second exoskeletal piece is configured to be attached to a second body location on the foot of the wearer proximate to the ankle joint;
wherein the first exoskeletal piece and the second exoskeletal piece move within a limiting factor of the first limiting connector, to allow natural motion of the ankle joint, and wherein the first exoskeletal piece and the second exoskeletal piece cannot move outside the limiting factor of the first limiting connector, to protect the ankle joint.

20. A shoe system for limiting body motion of an ankle joint of a wearer, the shoe system comprising:
a first limiting connector having a first endpoint and a second endpoint, wherein the second endpoint is configured to move with respect to the first endpoint within a limiting factor of the first limiting connector;
a first shoe piece coupled to the first endpoint, wherein the first shoe piece is configured to be attached around the leg of the wearer proximate to the ankle joint; and
a second shoe piece configured to be coupled to the second endpoint, wherein the second shoe piece is configured to be attached around the foot of the wearer proximate to the ankle joint;
wherein the first shoe piece and the second shoe piece move within a limiting factor of the first limiting connector, to allow natural motion of the ankle joint, and wherein the first shoe piece and the second shoe piece cannot move outside the limiting factor of the first limiting connector, to protect the ankle joint.
